(19) United States
(12) Patent Application Publication
(10) Pub. No.: US 2011/0143445 A1
(43) Pub. Date: Jun. 16, 2011

(54) Analysis of Amino Acids And Amine-Containing Compounds Using Tagging Reagents and LC-MS Workflow

(75) Inventors: Scott DANIELS, Northampton, MA (US); Subhasish PURKAYASTHA, Acton, MA (US)

(73) Assignee: DH Technologies Development Pte. Ltd., Singapore (SG)

(21) Appl. No.: 12/969,036

(22) Filed: Dec. 15, 2010

Related U.S. Application Data
(60) Provisional application No. 61/286,491, filed on Dec. 15, 2009.

(51) Int. Cl. G01N 33/68; C07D 403/12; G01N 1/28; G01N 1/34; G01N 33/00

(52) U.S. Cl. 436/90; 544/372; 436/174; 436/177; 436/111

(57) ABSTRACT

A plurality of mass differential tagging reagents is used to label amine functionality in amine-containing compounds. The labeled analytes have distinct retention times on a reversed phase column, and distinct masses. Under high energy collision, reporter groups can be generated and the intensity or the peak area detected for each reporter group can be used for quantitation. One exemplary set of reagents includes a set of three different mass differential reagents comprising tagging weights of 140 atomic mass units, 144 atomic mass units, and 148 atomic mass units, respectively, with reporter groups of 113, 117, and 121 atomic mass units, respectively. A package including each of the mass differential reagents is also provided and can include separate respective containers, for example, one for each of the different reagents. The package can also include one or more standards each comprising a respective known concentration of a respective known amine-containing compound.

### CROSS-REFERENCE TO RELATED APPLICATION

The present application claims a priority benefit from earlier filed U.S. Provisional Patent Application No. 61/286,491 filed Dec. 15, 2009, which is incorporated herein in its entirety by reference.

### FIELD

The present teachings relate to the fields of mass spectrometry and tagging reagents useful for mass spectrometry.

### BACKGROUND

Methods of analyzing amine-containing compounds have been known, however, it is desirable to provide a method for the relative and absolute quantitation of amine-containing compounds. Previous methods have exhibited low sensitivity, a need for ²H, ¹⁵C, or ¹⁵N isotope-containing amino acid standards, and/or a need for other isotope-labeled standards. A need exists for a method of quantitating amine-containing compounds that overcomes these drawbacks.

### SUMMARY

According to various embodiments, the methods of the present teachings utilize mass differential, mass spectrometry (MS) tagging reagents to label amine functionality of amine-containing compounds. The labeled analytes can have distinct retention times on a reversed phase column, and distinct masses. Under high energy collision, reporter groups can be generated. The intensity or the peak area detected for each reporter group can be used for quantitation.

A plurality of exemplary mass differential reagents that can be provided and/or used according to various embodiments of the present teachings are shown in FIGS. 1A-1I. One exemplary set of MS tagging reagents according to various embodiments of the present teachings comprises a set of three different mass differential reagents, for example, comprising a first reagent having a tagging weight of 140 atomic mass units, a second reagent having a tagging weight of 144 atomic mass units, and a third reagent having a tagging weight of 148 atomic mass units. The reporter ions in the MS/MS for these tags are 113, 117, and 121 atomic mass units, respectively. In some embodiments, such a set comprises the reagents shown in FIGS. 1A, 1E, and 1I, packaged together.

In some embodiments, a package including each of the different reagents is provided and can include separate respective containers, for example, one for each of the different reagents. One or more standards can also be provided, for example, each comprising a known concentration of a known amine-containing compound.

### BRIEF DESCRIPTION OF THE DRAWINGS

FIGS. 1A-1I show nine different reagents that are chemically identical, but differ from one another based on mass, and which can be used to form a set of tagging reagents.

FIG. 2 is a reaction scheme showing a general tagging reaction according to various embodiments of the present teachings.

FIG. 3 is a schematic flow chart showing the various steps involved with relative and absolute quantitation in a two-plex assay according to various embodiments of the present teachings.

FIG. 4 is a schematic flow chart showing the various steps involved with relative and absolute quantitation in a three-plex assay according to various embodiments of the present teachings.

FIG. 5 is a bar graph showing the precision and accuracy of a plasma control analysis according to various embodiments of the present teachings.

FIG. 6 is a bar graph showing a comparison of a plasma control in solution compared to plasma control by dried spot analysis protocol, according to various embodiments of the present teachings.

FIG. 7 is a bar graph showing the concentrations of each of three identical control plasma samples that were labeled with 115, 117, and 121 reagents and then mixed together with an internal standard, according to various embodiments of the present teachings.

FIG. 8 is a bar graph showing the precision and accuracy of urine control analysis according to various embodiments of the present teachings.

FIG. 9 is a bar graph showing the amount of the biogenic amines cadaverine, putrescine, phenylethylamine, and tyramine, and how they increase with increasing temperature, indicating spoilage.

### DETAILED DESCRIPTION

According to various embodiments, the present teachings provide a method for the quantitation of amine-containing compounds. While the method can be used for the quantitation of a wide variety of amine-containing compounds, the present teachings will be particularly exemplified with reference to the quantitation of amino acids. In some embodiments, the reagents and methods can be used for relative and absolute quantitation in two-plex, three-plex, and other multi-plex assays.

According to various embodiments, a plurality of mass spectrometry (MS) tagging reagents is provided for tagging one or more amine-containing compounds. The plurality can be packaged together as a set, packaged separately, or packaged in various combinations. The reagents can comprise a first tagging reagent having a chemical structure and a first mass. The chemical structure can comprise a moiety that is reactive to bond to a nitrogen atom of the amine functionality of the amine-containing compound. An exemplary reactive moiety can comprise an ester linkage to a carbonyl moiety. The nitrogen atom of the amine functionality of the amino-containing compound can react with the active ester of the tag to form an amide linkage to the tag. The hydrogen atom can be a hydrogen atom of a primary or secondary amine. Binding of the linkage can result in releasing a release moiety or leaving group comprising a hydroxylated moiety, for example, a hydroxylated succinimide.

The plurality of MS tagging reagents also comprises a second tagging reagent having the same chemical structure as the first tagging reagent but a different atomic mass compared to the first tagging reagent. The mass of the second tagging reagent can differ from that of the first tagging reagent by one or more atomic mass units. In an exemplary embodiment, the first tagging reagent can comprise, for example, a carbon atom, a nitrogen atom, a hydrogen atom, and/or an oxygen atom, but in the second tagging reagent the same carbon atom, nitrogen atom, hydrogen atom, or oxygen atom can be replaced by a ²H, ¹³C, a ¹⁵N, or an ¹⁸O isotope. If the chemical structure includes two carbon atoms, hydrogen atoms, and/or nitrogen atoms, and/or at least one oxygen atom, then the second tagging reagent can comprise two ²H, ¹³C or ¹⁵N isotopes, or one ¹⁸O isotope, and would thus have a mass of two atomic units over the mass of the first tagging reagent. In some embodiments, the first tagging reagent can comprise an isotope and the second tagging reagent can be free of that isotope, such that the first tagging reagent need not have the smallest mass of the plurality of tagging reagents. In some embodiments, each tagging reagent of the plurality comprises at least one isotope.

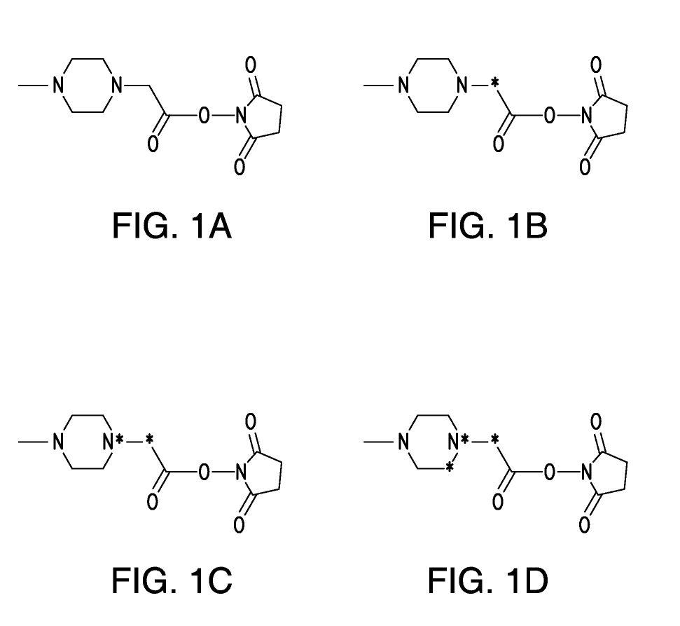

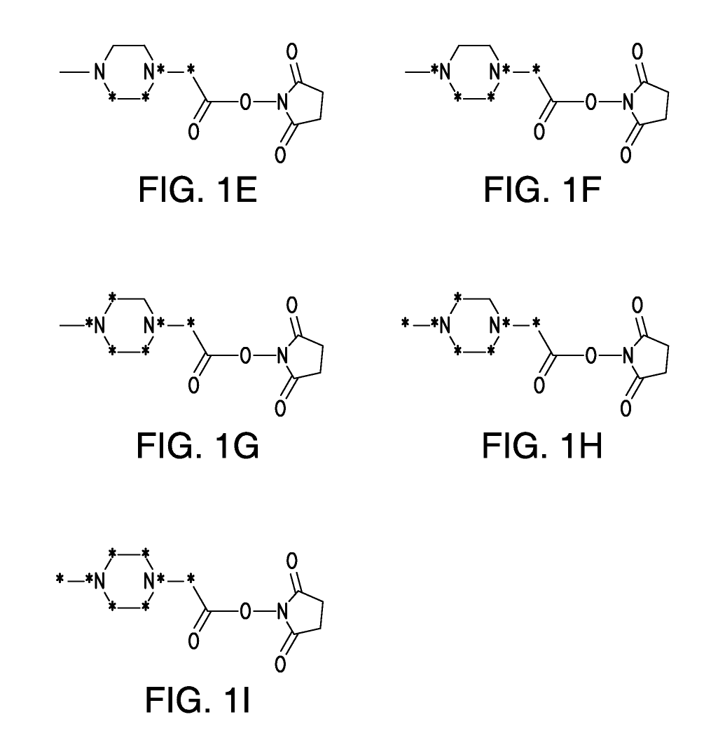

The plurality of MS tagging reagents can further comprise one or more additional tagging reagents, each having the same chemical structure as the first and second tagging reagents but each having a mass that differs from the mass of the first tagging reagent and the mass of the second tagging reagent, by one or more atomic mass units. An exemplary plurality of MS tagging reagents is shown in FIGS. 1A-1I, which show nine different MS tagging reagents, each having the same chemical structure as the others and each having a different atomic mass relative to the others. The tagging mass of the reagent shown in FIG. 1A is 140 atomic mass units (amu) and the tagging masses of the reagents shown in FIGS. 1B-1I go up by one amu each such that the tagging mass of the reagent shown in FIG. 1I is 148 amu. The mass of the reporter ions generated in the MS/MS fragmentation of a compound tagged with the reagent shown in FIG. 1A is 113 atomic mass units (amu) and the masses of the reporter ions generated in the MS/MS fragmentation of compounds tagged with the reagents shown in FIGS. 1B-1I go up by one amu each such that the tagging mass of the reporter ions generated from the reagent shown in FIG. 1I is 121 amu. As can be seen, the different weights can be attributed to the use of different isotopes.

According to various embodiments, a kit is provided for the quantitation of one or more amine-containing compounds. The kit can comprise one or more mass differential tagging reagents as described herein, for example, each stored in a separate respective container. In some embodiments, the kit can comprise a box, envelope, bag, or other outer container, inside of which can be the stored individual respective containers for the different tagging reagents. In some embodiments, the kit can comprise buffers and various reagents, useful to early out the methods. In some embodiments, the kit can comprise a plurality of MS tagging reagents wherein each of the tagging reagents have an atomic mass that differs from the atomic masses of the other tagging reagents by two or more atomic mass units. As an example, a kit can be provided that comprises the reagent shown in FIG. 1A, the reagent shown in FIG. 1E, and the reagent shown in FIG. 1I, which have tagging moiety masses of 140, 144, and 148 atomic mass units, respectively. In some embodiments, the plurality of tagging reagents can comprise two or more tagging reagents each having a mass that differs from the other reagents of the plurality by three or more atomic mass units, for example, by four or more atomic mass units.

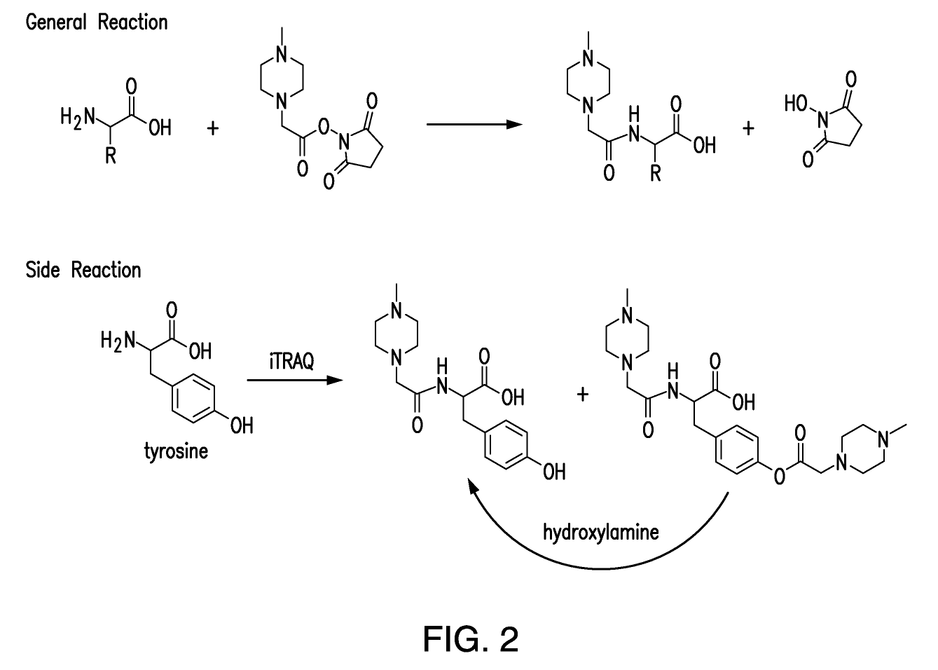

As shown in FIGS. 1A-1I, the plurality of MS tagging reagents can comprise differently weighted succinimide esters of an N-alkyl piperizene acetic acid, all having the same chemical structure. In the specific embodiment shown, each of the tagging reagents comprises N-hydroxy succinimide ester of N-methyl piperizene acetic acid. As an example of a tagging moiety, the N-methyl piperizene carbonyl moiety of the chemical structure is what reacts with and tags the amine-containing compound. A general reaction scheme of this exemplary tagging reaction, according to various embodiments, is shown in FIG. 2. The remainder of the chemical structure, along with the hydrogen obtained from the amine moiety of the amine-containing compound, is released or leaves as an N-hydroxy succinimide moiety. The moiety that is released during the tagging reaction is an example of what is referred to herein as the release moiety. The nitrogen atom of the amine functionality of the amino-containing compound can react with the active ester of the tag to form an amide linkage to the tag.

Other exemplary tagging reagents, tagging moieties, and release moieties that can be used in accordance with various embodiments of the present invention include those described, for example, in U.S. Pat. No. 7,195,751 B2 which is incorporated herein in its entirety by reference.

In use, a first tagging reagent of the plurality can be made to contact a standard that may, or may not, be included with the tagging reagents in a kit. The standard can comprise a known amine-containing compound, for example, a previously tagged amine-containing compound at a known concentration. The contact can be made under conditions that favor a reaction between the first tagging reagent and the standard. For example, the reaction can comprise a chemical reaction that binds the standard to the carbonyl N-alkyl piperizene moiety of the ester described above. The reaction can result in the release of the N-hydroxy succinimide moiety of the ester described above.

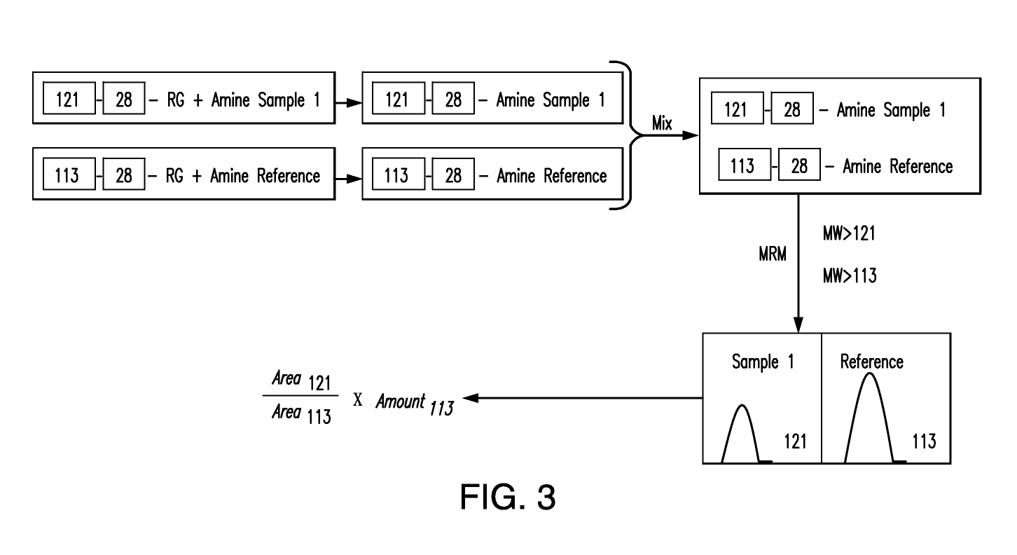

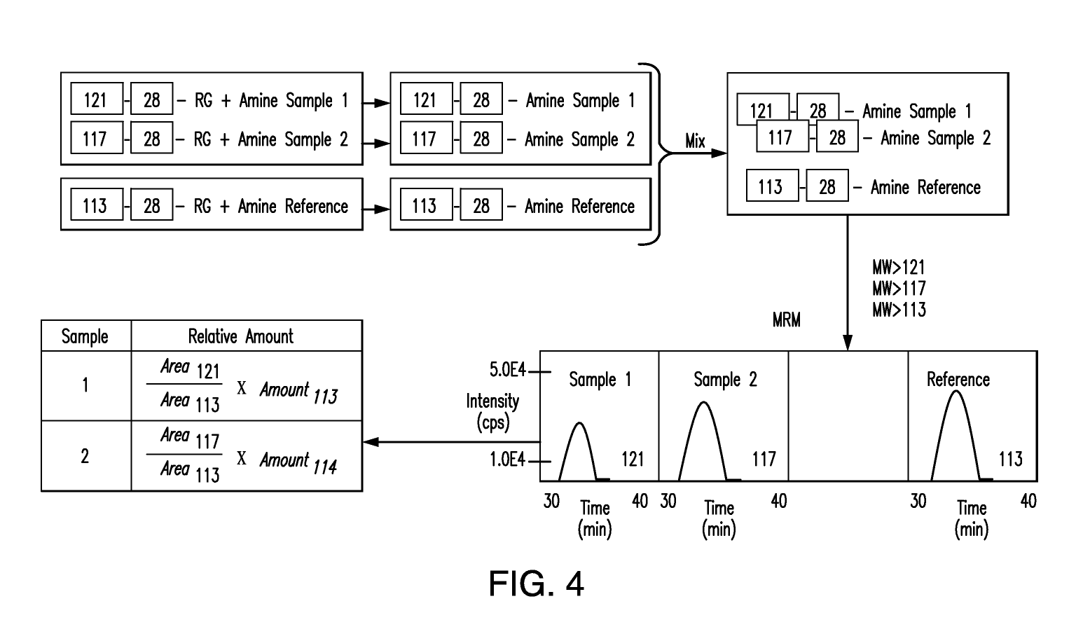

Also, when in use, a second tagging reagent of the plurality can be made to contact a sample comprising an unknown concentration of the same amine-containing compound. As described below with reference to FIG. 3, the tagged amine-containing compounds of the standard and sample can be mixed together and analyzed to determine the concentration of the amine-containing compound in the sample. The analysis can comprise separating the mixture to form separated analytes, and analyzing the separated analytes. Methods of separation that can be used include gas chromatographic methods, liquid chromatographic methods, HPLC methods, other chromatographic methods, electrophoretic methods, mass differential separation methods, and the like. In an exemplary embodiment, liquid chromatography is used to separate the various analytes in the mixture and thus form separated analytes. In some embodiments, chromatographic separation can be preformed on a reversed phase column and peaks eluting from the column can be subject to subsequent analysis. In some embodiments, the subsequent analysis can comprise mass spectrometry or, more particularly, Parent Daughter Ion Transition Monitoring (PDITM). By comparing the results from the PDITM, the concentration of the amine-containing compound in the sample can be determined, as is described in more detail with reference to FIGS. 3 and 4 below. More details about PDITM and its use can be found in published application US 2006/0183238 A1, which is incorporated herein in its entirety by reference.

An exemplary method of quantitation is shown with reference to FIG. 3, which illustrates relative and absolute quantitation for a two-plex assay. As described in FIG. 3, the method can begin with labeling a standard containing a known concentration of a known amino acid. The standard can be labeled with a first tagging reagent having the structure identified in FIG. 1A. The N-methyl piperizene moiety provides a tagging weight of 140 atomic mass units. Next, a sample to be tested is labeled with a second tagging reagent that is chemically identical to the first tagging reagent used to label the standard, but, the second tagging reagent has a different mass. In the example shown in FIG. 3, the second tagging reagent comprises the reagent shown in FIG. 1I, which contains isotopes ¹³C and ¹⁵N at the positions shown with asterisks. As can been seen by comparing the 140 amu mass of the reagent shown in FIG. 1A (having no isotopes) to the 148 amu tagging mass of the reagent shown in FIG. 1I (having eight isotopes), one can see how the tagging mass of the reagent of FIG. 1I has a mass that is eight atomic mass units greater than the tagging mass of the reagent of FIG. 1A. As mentioned above, the tagging reagents shown in FIGS. 1A-1I have tagging masses of 140 to 148 amu, respectively.

The next step of the method depicted in FIG. 3 comprises combining the labeled standard with the labeled test sample to form a mixture. Subsequently, the mixture is subjected to separation, such as liquid chromatography (LC) separation, for example, on a reversed phase column. In various embodiments, the mixture can be directly infused into a mass spectrometer, especially if there are a small number of analytes of interest having unique masses. The labeled analytes, here, tagged or labeled amino acids, elute from the column at separate times due to their different and distinct retention times on the column. The peaks eluted from the reversed phase column comprise peaks that contain the labeled analyte and the labeled standard. Next, each peak eluted from the column is subjected to Parent Daughter Ion Transition Monitoring (PDITM). The ratio of the signal intensity of peak area of the reporter signals generated from the labeled standard, relative to those generated from the labeled test sample, gives the relative concentration of the analyte in the test sample. When the concentration of the labeled standard is known, the specific concentration of the analyte in the sample can be determined.

According to various embodiments, a method is provided that can be used for the absolute quantitation of one or more amino acids, wherein standards having known concentrations of a plurality of known amino acids are used. In some embodiments, a kit or package is provided having a plurality of standards, one for each of a plurality of different amino acids sought to be tested in a sample.

Another exemplary method of relative and absolute quantitation is shown with reference to FIG. 4, which illustrates relative and absolute quantitation for a three-plea assay. As described in FIG. 4, the method can begin with labeling a reference or standard containing a known concentration of a known amino acid. The standard can be labeled with a first tagging reagent having the structure identified in FIG. 1A. The N-methyl piperizene moiety provides a tagging weight of 140 atomic mass units. Next, two amine-containing samples to be tested are labeled with a second and third tagging reagent, respectively, that are chemically identical to the first tagging reagent used to label the standard, but have different masses. In the example shown in FIG. 4, the second tagging reagent used to label Amine Sample 1 comprises the reagent shown in FIG. 1I, which contains isotopes ¹³C and ¹⁵N at the positions shown with asterisks. As can been seen by comparing the 140 amu tagging mass of the reagent shown in FIG. 1A (having no isotopes) to the 148 amu tagging mass of the reagent shown in FIG. 1I (having eight isotopes), one can see how the reagent of FIG. 1I has a mass that is eight atomic mass units greater than the reagent of FIG. 1A. The third tagging reagent used to label Amine Sample 2 comprises the reagent shown in FIG. 1E, which contains isotopes ¹³C and ¹⁵N at the positions shown with asterisks. As can been seen by comparing the 140 amu tagging mass of the reagent shown in FIG. 1A (having no isotopes) to the 144 amu tagging mass of the reagent shown in FIG. 1E (having four isotopes), one can see how the reagent of FIG. 1E has a mass that is four atomic mass units greater than the reagent of FIG. 1A. The next step of the method depicted in FIG. 4 comprises combining the labeled standard with the labeled test samples to form a mixture. Subsequently, the mixture is subjected to separation, such as liquid chromatography (LC) separation, for example, on a reversed phase column. In various embodiments, the mixture can be directly infused into a mass spectrometer, especially if there are a small number of analytes of interest having unique masses. The labeled analytes, here, tagged or labeled amino acids, elute from the column at separate times due to their different and distinct retention times on the column. The peaks eluted from the reversed phase column comprise peaks that contain the labeled analytes and the labeled standard. Next, each peak eluted from the column is subjected to Parent Daughter Ion Transition Monitoring (PDITM). The ratio of the signal intensity of peak area of the reporter signals generated from the labeled standard, relative to those generated from the labeled test samples, gives the relative concentrations of the analytes in the test samples. When the concentration of the labeled standard is known, the specific concentration of each analyte in each of the samples can be determined.

The tagging chemistry and the methodology of the present teachings provide increased sensitivity relative to known methods, and eliminate the need for ²H-containing, ¹⁵N-containing, N-containing, or ¹⁸O-containing amino acid standards. Each analyte can have its own internal standard. The reporter signals can be specific to the standard sample and to the test sample. By adding labeled calibration standard directly to the sample, the need to obtain a matrix that is free of endogenous analyte is eliminated. In some embodiments, using PDITM increases specificity and reduces the risk of error. The reagent design makes it a good tool for FlashQuant™ System application.

In some embodiments, the tagging chemistry and the method can be run on any triple quadrupole instruments or on any instrument with a MALDI source, for example, those including, but not limited to, an AB Sciex TripleTOF™ 5600 System, 5800 MALDI TOF/TOF™ System, 4800 MALDI TOF/TOF™ System, 4700 MALDI TOF/TOF™ System, or a FlashQuant™ System with a MALDI source. Reagent kits, data analysis software, and the MS platform can together be used as an analyzer system for amino acid analysis. The method can similarly be employed for other amine-containing compounds.

Different liquid chromatography and mass spectrometry methods, systems, and software that can be used in accordance with various embodiments of the present teachings include those described in U.S. Provisional Patent Application No. 61/182,748 filed May 31, 2009, and in U.S. Patent Application No. US 2006/0183238 A1 which published on Aug. 17, 2006. Both of these references are incorporated herein in their entireties by reference.

The present teachings will be more fully understood with reference to the following Examples that are intended to illustrate, not limit, the present teachings.

### EXAMPLES

### Sample Preparation (Reagent Labeling Protocol) Labeling a Physiological Sample (Plasma, Serum, Urine, Cerebrospinal Fluid)

### Precipitating Protein

40 μL of a physiological sample was transferred to a tube. 10 μL of Sulfosalicylic Acid containing 4000 pmol norleucine, was added. The tube was vortexed to mix, then spun at 10,000×g for 2 minutes. 10 μL of the supernatant was transferred to a clean tube.

Diluting with Labeling Buffer

40 μL of Labeling Buffer containing 800 pmol norvaline was added to the 10-μL aliquot of supernatant from above. The tube was vortexed to mix, then spun. 10 μL of the supernatant was transferred to a clean tube. This sample was labeled with a First Tagging Reagent (see Labeling Samples section below). The remaining supernatant was refrigerated.

### Prepare the Labeling Reagent Solution

Each vial containing the Tagging Reagent Δ8 was spun at room temperature to bring the solution to the bottom of the vial. Each tube was capped promptly. 70 μL of isopropanol was added to each. Each vial was dated. Each vial was vortexed to mix the solution, then spun.

### Labeling Samples

To the sample diluted supernatant from the “Diluting with labeling buffer” section above, 5 μL of the Tagging Reagent Δ8 solution was added. Unused Tagging Reagent Δ8 solution was stored at −15° C. or below. The tube was vortexed to mix then spun. The tube was incubated at room temperature for at least 30 min. Then, 5 μL of Hydroxylamine was added and the tube was again vortexed and spun. For unlabeled allo-isoleucine analysis, 5 μL of the diluted supernatant from “Diluting with labeling buffer” above, was added. The unlabeled internal standard norleucine from the Sulfosalicylic Acid reagent used for the allo-isoleucine analysis was already mixed with the sample. The sample was dried completely in a centrifugal vacuum concentrator for not more than one hour. The dried labeled samples were stored at −15° C. or below.

### Sample Preparation (Reagent Labeling Protocol) Labeling a Dried Blood Spot Sample

The blood samples were prepared by spotting seventy-five microliters of whole blood onto Whatman #903 sample collection paper, as per a typical collection protocol. A ⅛ inch punch from the dried blood filter paper (3 μL of whole blood equivalent).

### Precipitating Protein

187.5 μL of 80% acetonitrile was added to the tube and it was shaken for 30 min. 100 μL of the supernatant was transferred to a clean tube and it was dried.

Dissolution with Labeling Buffer

8 μL of Labeling Buffer containing 160 pmol norvaline was added to the dried supernatant from above. The tube was vortexed to mix, then spun.

### Prepare the Labeling Reagent Solution

Each vial containing the Tagging Reagent Δ8 was spun at room temperature to bring the solution to the bottom of the vial. Each tube was capped promptly. 70 μL of isopropanol was added to each. Each vial was dated. Each vial was vortexed to mix the solution, then spun.

### Labeling Samples

To the sample diluted supernatant from the “Diluting with labeling buffer” section above, 5 μL of the Tagging Reagent Δ8 solution was added. Unused Tagging Reagent Δ8 solution was stored at −15° C. or below. The tube was vortexed to mix then spun. The tube was incubated at room temperature for at least 30 min. Then, 5 μL of Hydroxylamine was added and the tube was again vortexed and spun. For unlabeled allo-isoleucine analysis, 5 μL of the diluted supernatant from “Diluting with labeling buffer” above, was added. The unlabeled internal standard norleucine from the Sulfosalicylic Acid reagent used for the allo-isoleucine analysis was already mixed with the sample. The sample was dried completely in a centrifugal vacuum concentrator for not more than one hour. The dried labeled samples were stored at −15° C. or below.

### Analysis of Labeled Amino Acids by LC/MS/MS

### Preparing the Internal Standard Solution

A vial of AA Internal Standard was spun to bring the lyophilized material to the bottom of the vial. The internal standard solution was prepared by reconstituting one vial of AA Internal Standard by: finding the amount of Standard Diluent that is specified on the AA Internal Standard vial label (approximately 1.8 mL); dispensing 1 mL of the Standard Diluent into the AA Internal Standard vial; vortexing the vial in 30- to 60-second increments until all material was dissolved; adding the remaining Standard Diluent (approximately 0.8 mL); and vortexing to mix.

### Adding the Internal Standard Solution

To the dried sample from the “Labeling samples” sections above, 32 μL of AA Internal Standard solution was added. The tube was vortexed to mix and then spun. The labeled sample/internal standard mixture was transferred to an autosampler vial with a low-volume insert. To remove potential air trapped in the bottom of the vial, the vial was tapped or spun.

### LC/MS/MS Analysis

The samples were run using the MS system-specific acquisition method. Each 2 μL injection contained Tagging Reagent Δ8-labeled amino acids in the sample and approximately 10 pmole of each Δ0-labeled amino acid (except 5 pmole cystine) from the AA Internal Standard. The sample also had 10 pmole of norleucine and norvaline. Norleucine was introduced into the sample during the precipitation step and was monitored to follow the recovery of amino acids from the precipitate. Norvaline was introduced into the sample during the labeling step and was monitored to check the efficiency of the labeling reaction.

### Mobile Phase Preparation

Mobile Phase A

For each liter of Mobile Phase A, 1 mL of Mobile Phase Modifier A was mixed with 100 μL of Mobile Phase Modifier B with 998.9 mL of Milli-Q water, or equivalent HPLC-grade water.

Mobile Phase B

For each 500 mL of Mobile Phase B, 0.5 mL of Mobile Phase Modifier A was mixed with 50 μL of Mobile Phase Modifier B with 499.5 mL of HPLC-grade methanol.

### HPLC Apparatus and Conditions

The following apparatus, parameters, and conditions were used:

Agilent 1100 system

Binary pump G1312A

Well-plate autosampler G1367A

Column oven G1316A

Micro vacuum degasser G1379B

Agilent 1200 system

Binary pump G1312A

Well-plate autosampler G1367B

Column oven G1316A

Micro vacuum degasser G1379B

Shimadzu Prominence system

System controller CBM-20A

2 Isocratic pumps LC-20AD (includes automatic purge kit and semi-micro gradient mixer SUS-20A)

On-line degasser DGU-20A3

Autosampler SIL-20AC

Column oven CTO-20AC

### Separation Column

AAA C18 reversed-phase column, 5 μM, 150×4.6 min

### Guard Column

None

### Mobile Phase A

Water+0.1% formic acid+0.01% heptafluorobutyric acid

### Mobile Phase B

Methanol+0.1% formic acid+0.01% heptafluorobutyric acid

### Gradient Profile

### Shimadzu Prominence

| Step | Total Time (min) | Module | Event | Parameter (%) |
|---|---|---|---|---|
| 1 | 0.30 | Pumps | Pump B Conc. | 2 |
| 2 | 6.00 | Pumps | Pump B Conc. | 40 |
| 3 | 10.00 | Pumps | Pump B Conc. | 40 |
| 4 | 11.00 | Pumps | Pump B Conc. | 90 |
| 5 | 12.00 | Pumps | Pump B Conc. | 90 |
| 6 | 13.00 | Pumps | Pump B Conc. | 2 |
| 7 | 18.00 | Controller | Stop | |

### Gradient

### Agilent 1100 and 1200 Series

| Total Time | Flow Rate | | |
|---|---|---|---|
| (min) | (μL/min) | A (%) | B (%) |
| 0.00 | 800 | 98.0 | 2.0 |
| 6.00 | 800 | 60.0 | 40.0 |
| 10.00 | 800 | 60.0 | 40.0 |
| 11.00 | 800 | 10.0 | 90.0 |
| 12.00 | 800 | 10.0 | 90.0 |
| 13.00 | 800 | 98.0 | 2.0 |
| 18.00 | 800 | 98.0 | 2.0 |

Flow Rate: 0.8 mL/min
Column oven temperature: 50° C.

### Injection Volume: 2 μL

### MS/MS Detection

MS/MS detection was optimized for the systems API 3200™, API 4000™, 3200 QTRAP®, and 4000 QTRAP® LC/MS/MS. The following conditions were used.

TurboIonSpray® ion source
Positive polarity
Scan type: MRM
Resolution Q1: unit
Resolution Q3: unit

### Ion Source/Gas and Compound Parameters

| | | | |
|---|---|---|---|
| API 3200 ™ | 3200 QTRAP ® | API 4000 ™ | 4000 QTRAP ® |
| System | System | System | System |

TurbolonSpray ® ion source/gas parameters

| | | | | |
|---|---|---|---|---|
| CUR | 20 | 20 | 20 | 20 |
| CAD | 3 | Medium | 3 | Medium |
| IS | 1500 | 1500 | 1500 | 1500 |
| TEM | 600 | 600 | 600 | 600 |
| GS 1 | 50 | 50 | 50 | 50 |
| GS 2 | 50 | 50 | 50 | 50 |
| ihe | On | On | On | On |

Compound parameters

| DP | 30 | 30 | 30 | 30 |
|---|---|---|---|---|
| EP | 10 | 10 | 10 | 10 |
| CE | See MRM Transitions, Retention Times, | | | |
| | and CE Values Table | | | |
| CXP | 5 | 5 | 5 | 5 |

### MRM Transitions, Retention Times, and CE Values

| | | | MH+ (amu) | | Retention | CE |
|---|---|---|---|---|---|---|
| Name | ID | Formula | Q1 | Q3 | Time (min) | Value |
| O-phospho- | PSer_IS | C₁₀H₂₀N₃O₇P | 326.11 | 113.1 | 2.1 | 30 |
| L-serine | PSer | C₄¹³C₆H₂₀N¹⁵N₂O₇P | 334.13 | 121.1 | | |
| O-phospho- | PEtN_IS | C₉H₂₀N₃O₅P | 282.12 | 113.1 | 2.3 | 30 |
| ethanolamine | PEtN | C₃¹³C₆H₂₀N¹⁵N₂O₅P | 290.14 | 121.1 | | |
| taurine | Tau_IS | C₉H₁₉N₃O₄S | 266.12 | 113.1 | 2.6 | 30 |
| | Tau | C₃¹³C₆H₁₉N¹⁵N₂O₄S | 274.13 | 121.1 | | |
| L-asparagine | Asn_IS | C₁₁H₂₀N₄O₄ | 273.16 | 113.1 | 4.6 | 30 |
| | Asn | C₅¹³C₆H₂₀N₂¹⁵N₂O₄ | 281.17 | 121.1 | | |
| L-serine | ser_IS | C₁₀H₁₉N₃O₄ | 246.15 | 113.1 | 4.8 | 30 |
| | Ser | C₄¹³C₆H₁₉N¹⁵N₂O₄ | 254.16 | 121.1 | | |
| glycine | Gly_IS | C₉H₁₇N₃O₃ | 216.13 | 113.1 | 5.0 | 30 |
| | Gly | C₃¹³C₆H₁₇N¹⁵N₂O₃ | 224.15 | 121.1 | | |
| hydroxy-L-proline | Hyp_IS | C₁₂H₂₁N₃O₄ | 272.16 | 113.1 | 5.0 | 30 |
| | Hyp | C₆¹³C₆H₂₁N¹⁵N₂O₄ | 280.18 | 121.1 | | |
| ethanolamine | EtN_IS | C₉H₁₉N₃O₂ | 202.16 | 113.1 | 5.2 | 30 |
| | ErN | C₃¹³C₆H₁₉N¹⁵N₂O₂ | 210.17 | 121.1 | | |
| L-glutamine | Gln_IS | C₁₂H₂₂N₄O₄ | 287.17 | 113.1 | 5.3 | 30 |
| | Gln | C₆¹³C₆H₂₂N₂¹⁵N₂O₄ | 295.19 | 121.1 | | |
| L-aspartic acid | Asp_IS | C₁₁H₁₉N₃O₅ | 274.14 | 113.1 | 5.6 | 30 |
| | Asp | C₅¹³C₆H₁₉N¹⁵N₂O₅ | 282.15 | 121.1 | | |
| L-citrulline | Cit_IS | C₁₃H₂₅N₅O₄ | 316.20 | 113.1 | 6.0 | 30 |
| | Cit | C₇¹³C₆H₂₅N₂¹⁵N₂O₄ | 324.21 | 121.1 | | |
| L-threonine | Thr_IS | C₁₁H₂₁N₃O₄ | 260.16 | 113.1 | 6.3 | 30 |
| | Thr | C₅¹³C₆H₂₁N¹⁵N₂O₄ | 268.18 | 121.1 | | |
| sarcosine | Sar_IS | C₁₀H₁₉N₃O₃ | 230.15 | 113.1 | 6.0 | 30 |
| | Sar | C₄¹³C₆H₁₉N¹⁵N₂O₃ | 238.16 | 121.1 | | |
| β-alanine | bAla_IS | C₁₀H₁₉N₃O₃ | 230.15 | 113.1 | 6.3 | 30 |
| | bAla | C₄¹³C₆H₁₉N¹⁵N₂O₃ | 238.16 | 121.1 | | |
| L-alanine | Ala_IS | C₁₀H₁₉N₃O₃ | 230.15 | 113.1 | 6.8 | 30 |
| | Ala | C₄¹³C₆H₁₉N¹⁵N₂O₃ | 238.16 | 121.1 | | |
| L-glutamic acid | Glu_IS | C₁₂H₂₁N₃O₅ | 288.16 | 113.1 | 6.5 | 30 |
| | Glu | C₆¹³C₆H₂₁N¹⁵N₂O₅ | 296.17 | 121.1 | | |
| L-histidine | His_IS | C₁₃H₂₁N₅O₃ | 296.17 | 113.1 | 6.5 | 30 |
| | His | C₇¹³C₆H₂₁N₃¹⁵N₂O₃ | 304.19 | 121.1 | | |
| 1-methyl- | 1MHis_IS | C₁₄H₂₃N₅O₃ | 310.19 | 113.1 | 6.3 | 30 |
| L-histidine | 1MHis | C₈¹³C₆H₂₃N₃¹⁵N₂O₃ | 318.20 | 121.1 | | |
| 3-methyl- | 3MHis_IS | C₁₄H₂₃N₅O₃ | 310.19 | 113.1 | 6.7 | 30 |
| L-histidine | 3MHis | C₈¹³C₆H₂₃N₃¹⁵N₂O₃ | 318.20 | 121.1 | | |
| argininosuccinic | Asa_IS | C₁₇H₃₀N₆O₇ | 431.24 | 113.1 | 7.0 | 50 |
| acid | Asa | C₁₁¹³C₆H₃₀N₄¹⁵N₂O₇ | 439.23 | 121.1 | | |
| homocitrulline | Hcit_IS | C₁₄H₂₇N₅O₄ | 330.21 | 113.1 | 7.1 | 30 |
| | Hcit | C₈¹³C₆H₂₇N₃¹⁵N₂O₄ | 338.23 | 121.1 | | |
| L-anserine | Ans_IS | C₁₇H₂₈N₆O₄ | 381.23 | 113.1 | 7.2 | 30 |
| | Ans | C₁₁¹³C₆H₂₈N₄¹⁵N₂O₄ | 389.24 | 121.1 | | |
| L-carnosine | Car_IS | C₁₆H₂₅N₆O₄ | 367.21 | 113.1 | 7.3 | 30 |
| | Car | C₁₀¹³C₆H₂₅N₄¹⁵N₂O₄ | 375.22 | 121.1 | | |
| L-α-amino-adipic | Aad_IS | C₁₃H₂₃N₃O₅ | 302.17 | 113.1 | 7.4 | 30 |
| acid | Aad | C₇¹³C₆H₂₃N¹⁵N₂O₅ | 310.19 | 121.1 | | |
| γ-amino-n-butyric | GABA_IS | C₁₁H₂₁N₃O₃ | 244.17 | 113.1 | 7.1 | 30 |
| acid | GABA | C₅¹³C₆H₂₁N¹⁵N₂O₃ | 252.18 | 121.1 | | |
| D,L-β-amino- | bAib_IS | C₁₁H₂₁N₃O₃ | 244.17 | 113.1 | 7.6 | 30 |
| isobutyric acid | bAib | C₅¹³C₆H₂₁N¹⁵N₂O₃ | 252.18 | 121.1 | | |
| L-α-amino-n- | Abu_IS | C₁₁H₂₁N₃O₃ | 244.17 | 113.1 | 7.9 | 30 |
| butyric acid | Abu | C₅¹³C₆H₂₁N¹⁵N₂O₃ | 252.18 | 121.1 | | |
| L-arginine | Arg_IS | C₁₃H₂₆N₆O₃ | 315.21 | 113.1 | 7.5 | 30 |
| | Arg | C₇¹³C₆H₂₆N₄¹⁵N₂O₃ | 323.23 | 121.1 | | |
| L-proline | Pro_IS | C₁₂H₂₁N₃O₃ | 256.17 | 113.1 | 7.6 | 30 |
| | Pro | C₆¹³C₆H₂₁N¹⁵N₂O₃ | 264.18 | 121.1 | | |
| L-ornithine | Orn_IS | C₁₂H₂₄N₄O₃ | 413.29 | 113.1 | 7.7 | 50 |
| | Orn | C₆¹³C₆H₂₄N₂¹⁵N₂O₃ | 429.32 | 121.1 | | |
| cystathionine | Cth_IS | C₁₄H₂₆N₄O₅S | 503.27 | 113.1 | 7.7 | 50 |
| | Cth | C₇¹³C₆H₂₆N₂¹⁵N₂O₅S | 519.29 | 121.1 | | |
| L-cystine | Cys_IS | C₁₃H₂₄N₄O₅S₂ | 521.22 | 113.1 | 7.7 | 50 |
| | Cys | C₇¹³C₆H₂₄N₂¹⁵N₂O₅S₂ | 537.25 | 121.1 | | |
| δ-hydroxylysine | Hyl_IS | C₁₁H₂₁N₃O₃ | 443.30 | 113.1 | 7.8 | 50 |
| | Hyl | C₅¹³C₆H₂₁N¹⁵N₂O₃ | 459.33 | 121.1 | | |
| L-lysine | Lys_IS | C₁₃H₂₆N₄O₃ | 427.30 | 113.1 | 8.0 | 50 |
| | Lys | C₇¹³C₆H₂₆N₂¹⁵N₂O₃ | 443.33 | 121.1 | | |
| L-methionine | Met_IS | C₁₂H₂₃N₃O₃S | 290.15 | 113.1 | 8.8 | 30 |
| | Met | C₆¹³C₆H₂₃N¹⁵N₂O₃S | 298.17 | 121.1 | | |
| L-valine | Val_IS | C₁₂H₂₃N₃O₃ | 258.18 | 113.1 | 8.9 | 30 |
| | Val | C₆¹³C₆H₂₃N¹⁵N₂O₃ | 266.20 | 121.1 | | |
| L-norvaline | Nva_IS | C₁₂H₂₃N₃O₃ | 258.18 | 113.1 | 9.2 | 30 |
| | Nva | C₆¹³C₆H₂₃N¹⁵N₂O₃ | 266.20 | 121.1 | | |
| L-tyrosine | Tyr_IS | C₁₆H₂₃N₃O₄ | 322.18 | 113.1 | 9.1 | 30 |
| | Tyr | C₁₀¹³C₆H₂₃N¹⁵N₂O₄ | 330.19 | 121.1 | | |
| L-homocystine | Hcy_IS | C₁₅H₂₈N₄O₅S₂ | 549.25 | 113.1 | 9.1 | 50 |
| | Hcy | C₉¹³C₆H₂₈N₂¹⁵N₂O₅S₂ | 565.28 | 121.1 | | |
| L-isoleucine | Ile_IS | C₁₃H₂₅N₃O₃ | 272.20 | 113.1 | 10.1 | 30 |
| | Ile | C₇¹³C₆H₂₅N¹⁵N₂O₃ | 280.21 | 121.1 | | |
| L-leucine | Leu_IS | C₁₃H₂₅N₃O₃ | 272.20 | 113.1 | 10.4 | 30 |
| | Leu | C₇¹³C₆H₂₅N¹⁵N₂O₃ | 280.21 | 121.1 | | |
| L-norleucine | Nle_IS | C₁₃H₂₅N₃O₃ | 272.20 | 113.1 | 10.6 | 30 |
| | Nle | C₇¹³C₆H₂₅N¹⁵N₂O₃ | 280.21 | 121.1 | | |
| L-phenylalanine | Phe_IS | C₁₆H₂₃N₃O₃ | 306.18 | 113.1 | 10.3 | 30 |
| | Phe | C₁₀¹³C₆H₂₃N¹⁵N₂O₃ | 314.20 | 121.1 | | |
| L-tryptophan | Trp_IS | C₁₈H₂₄N₄O₃ | 345.19 | 113.1 | 11.4 | 30 |
| | Trp | C₁₂¹³C₆H₂₄N₂¹⁵N₂O₃ | 353.21 | 121.1 | | |
| Unlabeled L-allo- | uNle_IS | C₅H₁₃NO₂ | 132.10 | 86.1 | 8.5 | 18 |
| isoleucine | alloIle | C₆H₁₃NO₂ | 132.10 | 86.1 | 7.9 | |
| Unlabeled L- | uNle_IS | C₆H₁₃NO₂ | 132.10 | 86.1 | 8.5 | 18 |
| isoleucine | uIle | C₅H₁₃NO₂ | 132.10 | 86.1 | 8.1 | |
| Unlabeled L- | uNle_IS | C₅H₁₃NO₂ | 132.10 | 86.1 | 8.5 | 18 |
| leucine | uLeu | C₆H₁₃NO₂ | 132.10 | 86.1 | 8.4 | |

Dynamic Range Using the aTRAQ™ Kit (on the 3200 QTRAP® System)

| Amino | LLOQ | ULOQ | Orders of | Correlation |
|---|---|---|---|---|
| Acid | (μM) | (μM) | Magnitude | Coefficient |
| 1MHis | 0.2 | >10000 | 4.7 | 1.000 |
| 3MHis | 0.2 | >10000 | 4.7 | 0.997 |
| Aad | 0.2 | >10000 | 4.7 | 1.000 |
| Abu | 0.5 | >10000 | 4.3 | 1.000 |
| Ala | 0.2 | >10000 | 4.7 | 0.996 |
| Ans | 0.2 | >10000 | 4.7 | 0.997 |
| Arg | 0.5 | >10000 | 4.3 | 0.999 |
| Asa | 1.0 | >10000 | 4.0 | 0.999 |
| Asn | 0.5 | >10000 | 4.3 | 1.000 |
| Asp | 0.1 | >10000 | 5.0 | 0.996 |
| bAib | 0.1 | >10000 | 5.0 | 1.000 |
| bAla | 0.5 | >10000 | 4.3 | 1.000 |
| Car | 0.5 | >10000 | 4.3 | 1.000 |
| Cit | 0.5 | >10000 | 4.3 | 0.999 |
| Cth | 0.5 | >10000 | 4.3 | 1.000 |
| Cys | 1.0 | >10000 | 4.0 | 0.999 |
| EtN | 0.1 | >10000 | 5.0 | 1.000 |
| GABA | 0.1 | >10000 | 5.3 | 0.998 |
| Gln | 0.5 | >10000 | 4.3 | 0.999 |
| Glu | 0.5 | >10000 | 4.3 | 0.999 |
| Gly | 1.0 | >10000 | 4.0 | 1.000 |
| Hcit | 0.2 | >10000 | 4.7 | 1.000 |
| Hcy | 0.5 | >10000 | 4.3 | 0.999 |
| His | 0.5 | >10000 | 4.3 | 1.000 |
| Hyl | 0.5 | >10000 | 4.3 | 1.000 |
| Hyp | 0.2 | >10000 | 4.7 | 1.000 |
| Ile | 0.5 | >10000 | 4.3 | 1.000 |
| Leu | 0.5 | >10000 | 4.3 | 1.000 |
| Lys | 0.5 | >10000 | 4.3 | 0.999 |
| Met | 0.1 | >10000 | 5.0 | 1.000 |
| Nle | 0.2 | >10000 | 4.7 | 1.000 |
| Nva | 0.2 | >10000 | 4.7 | 0.999 |
| Orn | 0.5 | >10000 | 4.3 | 0.999 |
| PEtN | 0.5 | >10000 | 4.3 | 1.000 |
| Phe | 0.2 | >10000 | 4.7 | 0.999 |
| Pro | 0.1 | >10000 | 5.0 | 1.000 |
| PSer | 0.5 | >10000 | 4.3 | 0.995 |
| Sar | 0.2 | >10000 | 4.7 | 1.000 |
| Ser | 0.5 | >10000 | 4.3 | 1.000 |
| Tau | 0.5 | >10000 | 4.3 | 0.997 |
| Thr | 0.2 | >10000 | 4.7 | 0.998 |
| Trp | 0.1 | >10000 | 5.0 | 1.000 |
| Tyr | 0.5 | >10000 | 4.3 | 0.999 |
| Val | 0.2 | >10000 | 4.7 | 1.000 |

The accuracy of each amino acid determination was calculated from 0.01 μM to 10,000 μM. The dynamic range was set where all the accuracies were between 80% and 120%. The dynamic range was ≦1 to ≧10,000 μM.

### Precision and Accuracy of Plasma Control Analysis

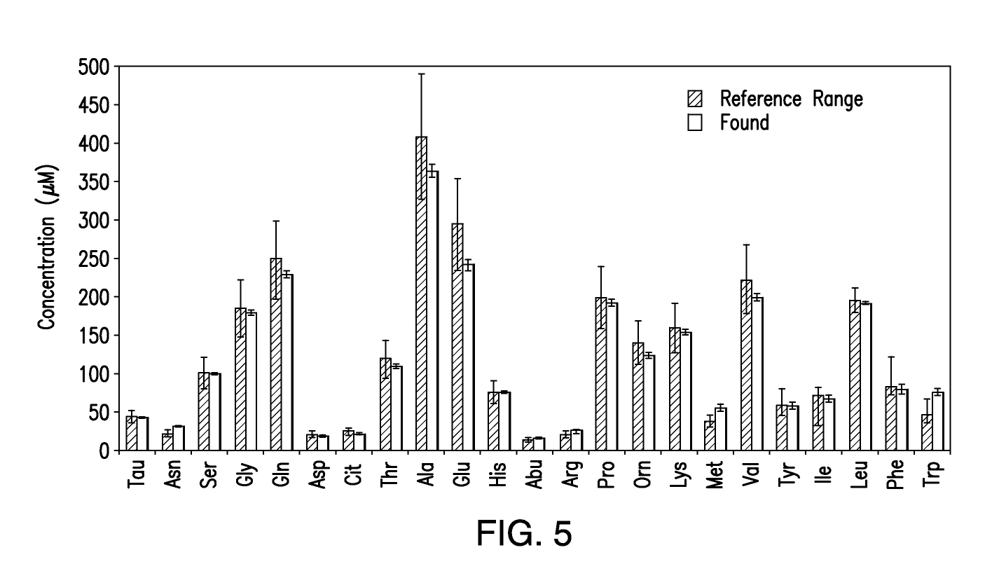

The Control Plasma sample was characterized using conventional ninhydrin amino acid analysis methods to determine a reference range. The aTRAQ method gave an average accuracy of 103.2% with an average % CV of 2.9%. The least accurate amino acids (Asn, Met, and Trp) are those that can sometimes present problems in conventional amino acid analysis. The resulting data is shown in FIG. 5. The data is from 10 runs (2 labelings with multiple runs of each sample).

### Plasma Control in Solution Compared to Plasma Control by Dried Spot Analysis Protocol

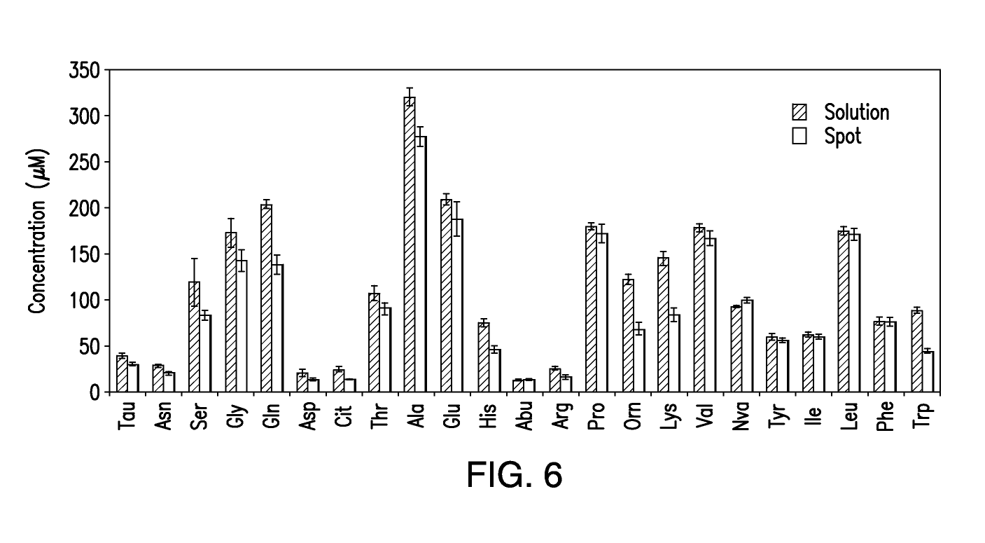

The Control Plasma was used to validate the alternate sample preparation method used for samples dried on Whatman #903 sample collection paper (i.e. pediatric blood spots). A punch out ⅛″ disc of each spotted sample (3 ul) was analyzed using the aTRAQ™ kit with an internal standard for every amino acid. The standard solution method and alternate spot method were run in parallel. The resulting data is shown in FIG. 6. This data represents three replicate labeling preparations (3 punch outs) with each analyzed by LC/MS/MS in triplicate. FIG. 6 shows the concentration of each amino acid for each method. This data shows a good correlation between the solution method and spot method of analysis.

### Multiplex Analysis of Control Plasma

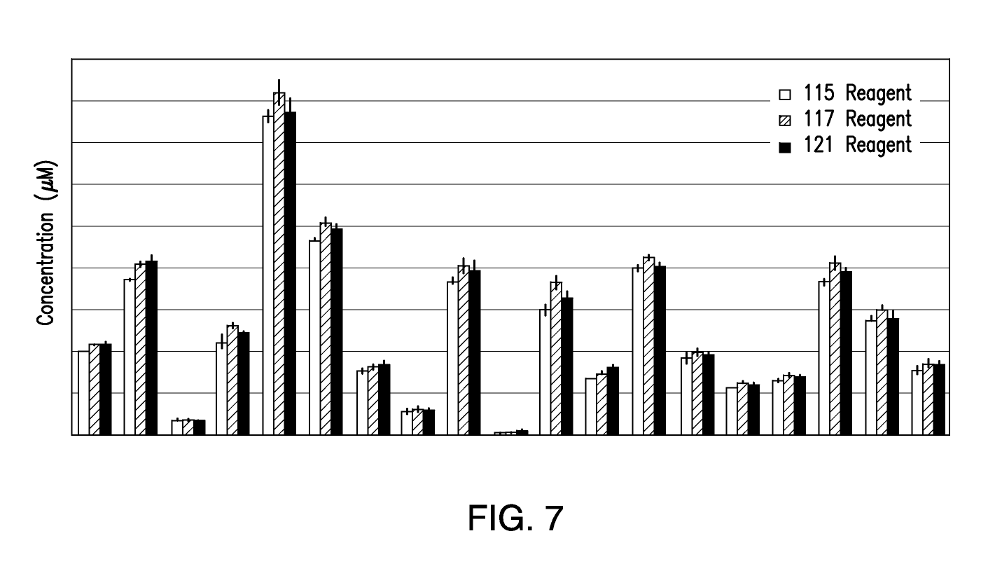

Three identical Control Plasma samples were labeled with the 115, 117, and 121 reagents and then mixed together with the internal standard labeled with the 113 reagent. The single mixed sample was analyzed and the concentrations for each sample determined. The results are shown in FIG. 7. The results show good agreement between the samples labeled with the different reagents.

### Precision and Accuracy of Urine Control Analysis

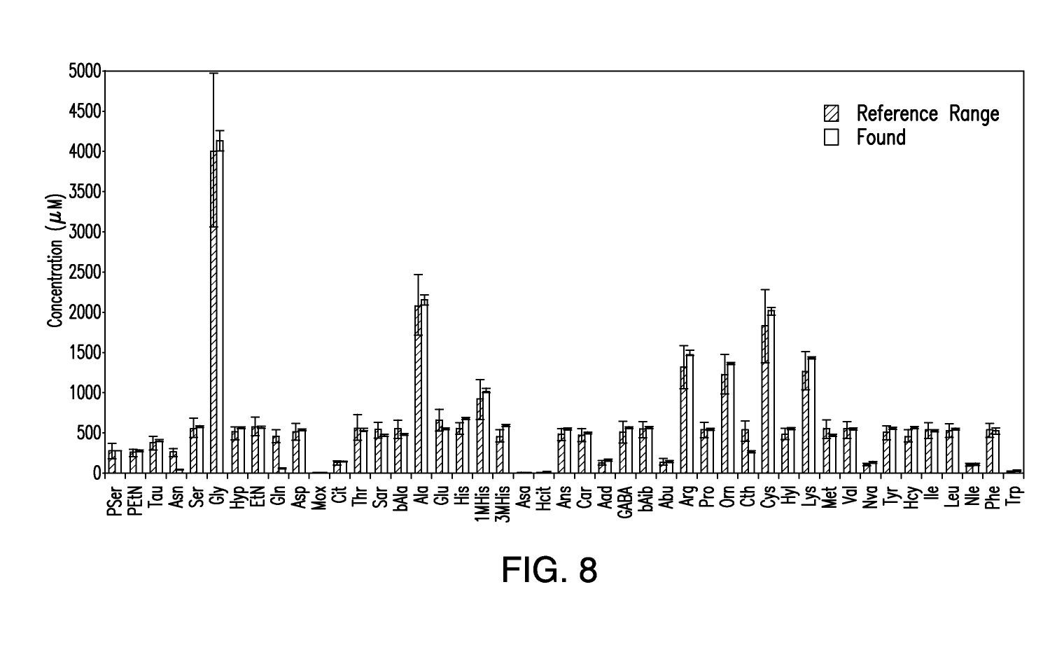

The Urine Control sample is a urine matrix into which amino acids have been spiked to known levels. The aTRAQ method gave an average accuracy of 103.3% with an average % CV of 2.7%. The resulting data is shown in FIG. 8. The data is from 10 runs (2 labelings and multiple runs of each sample).

### Biogenic Amine Amounts in Media Samples

| | Amount (mg/L) | | | |
|---|---|---|---|---|
| | CHO | FortiCHO | OptiCHO | Hybridoma |
| Ethanolamine | 13.3 | 37.4 | 7.37 | 2.24 |
| Histamine | 0 | 0 | 0 | 0 |
| Putrescine | 0.388 | 0.506 | 0.194 | 0.056 |
| Spermidine | 0 | 0 | 0 | 0 |
| Spermine | 19.8 | 18.8 | 5.45 | 0.261 |
| Cadaverine | 0 | 0 | 0 | 0 |
| Serotonin | 0 | 0 | 0 | 0 |
| Diaminoheptane | 0 | 0 | 0 | 0 |
| Tyramine | 0 | 0 | 0 | 0 |
| Phenylethylamine | 0.0318 | 0 | 0 | 0 |
| Tryptamine | 0 | 0 | 0 | 0 |

As can be seen from the table above, the theoretical values in the CHO sample for ethanolamine, putrescine, and spermine are 13.6, 0.543, and 15.6 mg/L, respectively.

### Salmon Spoilage—Biogenic Amine Concentrations at Different Storage Conditions

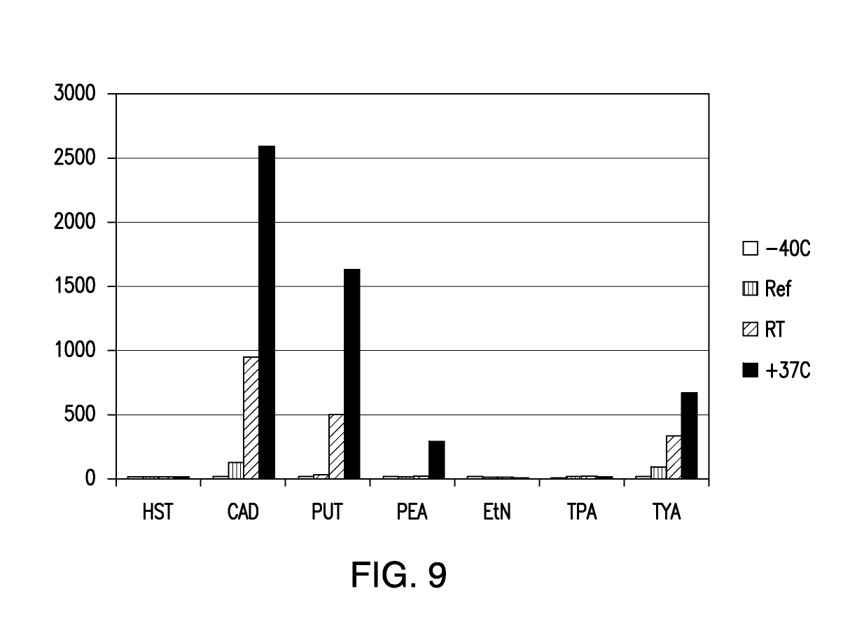

A sample of salmon was stored at different temperatures for 3 days and then labeled with the aTRAQ™ reagent and the amount of biogenic amine was determined. The results are shown in FIG. 9. As can be seen, the amount of some of the biogenic amines (cadaverine, putrescine, phenylethylamine, and tyramine) increase with increasing temperature, indicating spoilage.

Other embodiments of the present teachings will be apparent to those skilled in the art from consideration of the present specification and practice of the present teachings disclosed herein. It is intended that the present specification and examples be considered exemplary only.

What is claimed:

1. A plurality of mass spectrometry (MS) tagging reagents for tagging one or more amine-containing compounds, the plurality of MS tagging reagents comprising:
a first tagging reagent having a chemical structure and a first mass, the chemical structure including a moiety that is reactive to bind to a nitrogen atom of an amine functionality of an amine-containing compound; and
a second tagging reagent having the same chemical structure as the first tagging reagent and a second mass that is different than the first mass by one or more atomic mass units.

2. The plurality of MS tagging reagents of claim 1, further comprising at least a third tagging reagent having the same chemical structure as the first tagging reagent and a third mass that differs from the first mass and the second mass by one or more atomic mass units.

3. The plurality of MS tagging reagents of claim 1, wherein the first tagging reagent is disposed within a first container, the second tagging reagent is disposed within a second container separate from the first container, and both the first container and the second container are disposed within a third container.

4. The plurality of MS tagging reagents of claim 1, wherein the second mass differs from the first mass by two or more atomic mass units.

5. The plurality of MS tagging reagents of claim 1, wherein the second mass differs from the first mass by three or more atomic mass units, and the third mass differs from the second mass by three or more atomic mass units.

6. The plurality of MS tagging reagents of claim 1, wherein the first tagging reagent is in contact with a standard comprising a known concentration of an amine-containing compound and the second tagging reagent is in contact with a sample comprising an unknown concentration of the amine-containing compound.

7. The plurality of MS tagging reagents of claim 1, wherein the first tagging reagent comprises a succinimide ester of an N-alkyl piperizene acetic acid.

8. The plurality of MS tagging reagents of claim 7, wherein the succinimide ester of an N-alkyl piperizene acetic acid comprises N-hydroxy succinimide ester of N-methyl piperizene acetic acid.

9. The plurality of MS tagging reagents of claim 7, wherein the second tagging reagent comprises at least one carbon atom that is a ¹³C isotope.

10. The plurality of MS tagging reagents of claim 7, wherein the second tagging reagent comprises at least one nitrogen atom that is a ¹⁵N isotope.

11. The plurality of MS tagging reagents of claim 7, wherein the second tagging reagent comprises at least one oxygen atom that is an ¹⁸O isotope.

12. The plurality of MS tagging reagents of claim 7, wherein the second tagging reagent comprises at least one hydrogen atom that is a ²H isotope.

13. A method comprising:
contacting a standard comprising an amine-containing compound having a known structure with a first mass spectrometry (MS) tagging reagent, the first MS tagging reagent having a chemical structure, and a first mass, the chemical structure including a moiety that is reactive to bind to a nitrogen atom of an amine functionality of an amine-containing compound; and
contacting a sample comprising the amine-containing compound with a second MS tagging reagent, the second MS tagging reagent having the same chemical structure as the first MS tagging reagent and a second mass that differs from the first mass by one or more atomic mass units.

14. The method of claim 13, wherein the standard has a known concentration of the amine-containing compound, and the sample has an unknown concentration of the amine-containing compound.

15. The method of claim 13, further comprising mixing together the standard in contact with the first MS tagging reagent, or a reaction product thereof, with the sample in contact with the second MS tagging reagent, or a reaction product thereof, to form a mixture.

16. The method of claim 15, further comprising subjecting the mixture to liquid chromatographic (LC) separation to form separated analytes.

17. The method of claim 16, further comprising eluting the separated analytes to form eluting peaks and subjecting the eluting peaks to mass spectrometry analysis.

18. The method of claim 16, further comprising eluting the separated analytes to form eluting peaks and subjecting the eluting peaks to parent daughter ion transition monitoring (PDITM).

19. The method of claim 18, further comprising comparing results from the PDITM and, based on the comparison, determining the concentration of the amine-containing compound in the sample.

20. The method of claim 15, further comprising subjecting the mixture to a two-plex assay.

21. The method of claim 15, further comprising subjecting the mixture to a multi-plex assay.

22. The method of claim 13, wherein the chemical structure comprises a tagging moiety and a release moiety, the chemical structure comprises a linkage between the tagging moiety and the release moiety, and the method further comprises binding the tagging moiety to a nitrogen atom of the amine-containing compound, at the linkage, and releasing the release moiety.

23. The method of claim 15, further comprising directly infusing the mixture into a mass spectrometer.
